(19) United States
(12) Patent Application Publication
(10) Pub. No.: US 2016/0002675 A1
(43) Pub. Date: Jan. 7, 2016

(54) METHOD FOR PRODUCING ETHANOL FROM CELLULOSIC BIOMASS

(71) Applicant: COSMO OIL CO., LTD., Minato-ku (JP)

(72) Inventors: Hiroshi NAGASAKI, Satte-shi (JP); Yuya SUZUKI, Satte-shi (JP)

(73) Assignee: COSMO OIL CO., LTD., Minato-ku (JP)

(21) Appl. No.: 14/776,136

(22) PCT Filed: Feb. 28, 2014
(86) PCT No.: PCT/JP2014/054995

§ 371 Date: Sep. 14, 2015

(30) Foreign Application Priority Data
Mar. 15, 2013 (JP) 2013-052869

(51) Int. Cl. C12P 7/10

(52) U.S. Cl. CPC C12P 7/10

(57) ABSTRACT

Provided is a method for efficiently producing ethanol even when a fermentation inhibitor is present in a cellulosic biomass hydrolysate.

A method for producing ethanol comprising fermenting a fermentation broth comprising a cellulosic biomass hydrolysate using an yeast belonging to Candida intermedia under a condition such that an air supply rate into a fermenter is from 0.0001 to 100 L/hour/g dry cell weight.

### TECHNICAL FIELD

The present invention relates to a method for producing ethanol by alcohol fermentation using cellulosic biomass hydrolysate.

### BACKGROUND ART

Cellulosic biomass has drawn attention as an ethanol production raw material by microbial fermentation in view of environmental issues. Particularly, from a viewpoint of utilizing unused biomass, use of cellulosic biomass raw materials from wood, papers, or agricultural wastes such as bagasse (cane trash), corn stover (core, stalk, leaves, etc. of corn) as well as straws has been studied in recent years (Patent Literatures 1 to 3).

Meanwhile, for the production of ethanol using a cellulosic biomass by microbial fermentation, it is required to decompose cellulose, hemicellulose and polysaccharides, which are partial decomposed material thereof, contained in cellulosic biomass, to obtain a saccharified solution containing as principal components hexose such as glucose, mannose and galactose or pentose such as xylose and subject the sugar in the saccharified solution to the microbial fermentation. Further, enzymatic methods and hydrolysis methods such as dilute sulfuric acid method and hydrothermal treatment are known as the methods for decomposing such a cellulosic biomass. Of these methods, the saccharification caused by the enzymatic method does not produce furan compounds such as furfural and 5-hydroxymethylfurfural (HMF) or excessively decomposed products of acetic acid, formic acid or levulinic acid in the saccharified solution but requires a large amount of many different species of enzymes, hence posing a cost problem for industrialization. Meanwhile, the hydrolysis methods such as dilute sulfuric acid method and hydrothermal treatment are advantageous costwise but produce furan compounds such as furfural and 5-hydroxymethylfurfural (HMF) and various excessively decomposed products (byproducts) such as weak acids including acetic acid, formic acid or levulinic acid, and these byproducts are known to inhibit the ethanol production from monosaccharides (Non Patent Literatures 1 to 3).

### CITATION LIST

### Patent Literature

[Patent Literature 1] JP-A-2006-88136
[Patent Literature 2] JP-A-2008-54676
[Patent Literature 3] JP-A-2008-260811

### Non Patent Literature

[Non Patent Literature 1] A. Petersson et al., “A 5-hydroxymethyl furfural reducing enzymes encoded by the Saccharomyces cerevisiae ADH6 gene conveys HMF tolerance”, Yeast, 2006, Vol. 23, p. 455-464
[Non Patent Literature 2] J. A. van Maris et al., “Alcoholic fermentation of carbon sources in biomass hydrolysates by Saccharomyces cerevisiae”, Antonie van Leeusenhoek, 2006, Vol. 90, p. 391-418
[Non Patent Literature 3] E. Palmqvist and B. Hahn-Hagardal, “Fermentation of lignocellulosic hydrolysate. II: inhibitors and mechanisms of inhibition”, Bioresource Technology, 2000, Vol. 74, p. 25-33

### SUMMARY OF INVENTION

An object of the present invention is to provide a method for efficiently producing ethanol even when a fermentation inhibitor is present in a cellulosic biomass hydrolysate.

The present inventors conducted extensive studies to solve the above problems and found that, in a method for producing ethanol using a fermentation broth comprising a cellulosic biomass hydrolysate, an efficient ethanol production can be continued even when a fermentation inhibitor originated from a cellulosic biomass hydrolysate is present in the fermentation broth when the fermentation is carried out using an yeast belonging to Candida intermedia under a predetermined aeration condition, whereby the present invention was accomplished.

More specifically, the present invention provides the following [1] to [3].

[1] A method for producing ethanol comprising fermenting a fermentation broth comprising a cellulosic biomass hydrolysate using an yeast belonging to Candida intermedia under a condition such that an air supply rate into a fermenter is from 0.0001 to 100 L/hour/g dry cell weight.
[2] The method for producing ethanol according to [1], wherein the fermentation is a continuous method in which the fermentation broth comprising a cellulosic biomass hydrolysate is supplied into the fermenter at a supply rate of from 0.0002 to 2 L/hour/g dry cell weight.
[3] The method for producing ethanol according to [1] or [2], wherein the yeast belonging to Candida intermedia is an yeast designated as 4-6-4T2 and deposited under FERM BP-11509.

### Advantageous Effect of Invention

According to the method for producing ethanol of the present invention, ethanol can be efficiently produced from a cellulosic biomass hydrolysate in which a fermentation inhibitor is present.

### BRIEF DESCRIPTION OF DRAWINGS

FIG. 1 is a drawing showing the time dependent changes in the ethanol concentrations when 0.5% by mass of acetic acid (0.08 mol/L) was added to a fermentation broth to produce ethanol by each of the yeasts.

FIG. 2 is a drawing showing the time dependent changes in the ethanol concentrations when 0.3% by mass of acetic acid (0.05 mol/L), 0.1% by mass of furfural (0.010 mol/L) and 0.1% by mass of HMF (0.008 mol/L) were added to a fermentation broth to produce ethanol by each of the yeasts.

FIG. 3 is a drawing showing the time dependent changes in the ethanol concentrations when 0.3% by mass of acetic acid (0.050 mol/L), 0.3% by mass of levulinic acid (0.026 mol/L) and 0.2% by mass of formic acid (0.043 mol/L) were added to a fermentation broth to produce ethanol by each of the yeasts.

FIG. 4 is a drawing showing the time dependent changes in the ethanol concentrations when 0.22% by mass of furfural (0.023 mol/L) and 0.68% by mass of HMF (0.054 mol/L) were added to a fermentation broth to produce ethanol by Candida intermedia 4-6-4T2 (FERM BP-11509).

FIG. 5 is a drawing showing the time dependent changes in the ethanol production concentration by continuous fermentation using Candida intermedia NBRC10601.

FIG. 6 is a drawing showing the time dependent changes in the ethanol production concentration by continuous fermentation using Candida intermedia 4-6-4T2 (FERM BP-11509).

### DESCRIPTION OF EMBODIMENTS

### (1) Yeast

The method for producing ethanol of the present invention uses an yeast belonging to Candida intermedia. The yeast is not particularly limited as long as it belongs to Candida intermedia and may be, for example, Candida intermedia “NBRC10601” obtainable from National Institute of Technology and Evaluation or a mutant of Candida intermedia. Of these, it is particularly preferable to use the yeast, which was obtained when the present inventors naturally mutated using Candida intermedia “NBRC10601” as a parental strain in accordance with a routine method, selected strains having higher ethanol productivity than the parental strain, and was designated as Candida intermedia “4-6-4T2” and deposited under “FERM BP-11509” in National Institute of Technology and Evaluation, International Patent Organisms Depositary (NITE) (1-1-1 Central 6, Higashi, Tsukuba, Ibaraki, Japan) (the original deposit date, Sep. 6, 2011).

The above 4-6-4T2, in the co-presence of glucose and xylose, has an ability to efficiently produce ethanol in a short period of time from glucose and xylose. In the co-presence of glucose and xylose herein means that 4-6-4T2 is co-present in the raw material liquid containing at least glucose and xylose (fermentation broth). The conventional yeasts are, as described above, those with insufficient xylose consumption efficiency, or those with an ethanol production ability from either one of glucose or xylose but in which no substantial xylose consumption until glucose is thoroughly consumed due to the catabolite repression when both glucose and xylose are present, but, 4-6-4T2, even when both glucose and xylose are present, has an ability to efficiently produce ethanol in a short period of time from both of them.

Further, 4-6-4T2 efficiently produces ethanol in a short period of time from a raw material liquid containing glucose and xylose but xylitol is not substantially produced as a byproduct from this process. Furthermore, 4-6-4T2 has properties equivalent to those of the parental strain thereof except the ethanol production ability from these saccharides.

Pichia stipitis, Candida shehatae and Pachysolen tannophilus are also known as the yeasts capable of producing ethanol from a cellulosic biomass hydrolysate. These yeasts can produce ethanol by consuming xylose, which is hard to consume among the saccharides contained in a cellulosic biomass hydrolysate. However, these yeasts fail to produce enough ethanol in the presence of a fermentation inhibitor contained in a cellulosic biomass hydrolysate. For this reason, even when these yeasts are replaced with an yeast belonging to Candida intermedia used in the present invention, efficient ethanol production cannot be achieved, unlike the present invention.

### (2) Fermentation Broth Comprising a Cellulosic Biomass Hydrolysate

According to the method for producing ethanol of the present invention, a cellulosic biomass hydrolysate is contained as a carbon source.

The cellulosic biomass used herein refers to the biomass which encompasses cellulose and hemicellulose. When cellulose contained in the biomass is hydrolyzed, glucose is obtained, whereas when the hemicellulose is hydrolyzed, glucose, xylose, mannose and galactose are obtained. The content ratio of each saccharide contained in the cellulosic biomass hydrolysate varies depending on the kind of cellulosic biomass, but each of them contains glucose, xylose, mannose and galactose.

According to the present invention, biomass obtained from agricultural residues (rice straw, wheat straw, bagasse, cone stover, and the like), forestry residues (timber, and the like) and the like is preferably used as the cellulosic biomass, which is a raw material for the hydrolysis from a viewpoint of the economic advantage in the ethanol production.

The cellulosic biomass hydrolysate used in the present invention refers to those obtained by hydrolyzing a cellulosic biomass using a dilute sulfuric acid method or a hydrothermal treatment. For example, in the dilute sulfuric acid method a cellulosic biomass is dried and crushed, and subsequently distilled water or sulfuric acid (0.2 to 0.5% by mass) is added thereto so that the weight is 10 times the amount to carry out hydrolysis at 190° C. to 210° C. for 5 to 10 minutes (P-M. Bondesson et al., “Ethanol and biogas production after steam pretreatment of corn stover with or without the addition of sulphuric acid”, Biotechnol., for Biofuel. 2013, Vol. 6, p. 1-11), or in the hydrothermal treatment a cellulosic biomass is hydrolyzed at 190° C. to 210° C. for 5 to 10 minutes to obtain a saccharified solution suitable for the ethanol production (the same Literature as above).

A cellulosic biomass hydrolysate contains furan compounds such as furfural and 5-hydroxymethylfurfural (HMF) and weak acids such as acetic acid, formic acid and levulinic acid as fermentation inhibitors produced during the hydrolysis treatment. A typical content of the representative fermentation inhibitor in a cellulosic biomass hydrolysate is, for example, 0.0 to 0.05 mol/L of furfural or HMF and about 0.0 to 0.15 mol/L of acetic acid, formic acid or levulinic acid (H. B. Klinke et al., “Inhibition of ethanol-producing yeast and bacteria by degradation products produced during pre-treatment of biomass”, Appl. Microbiol. Biotechnol. Vol. 66, p. 10-26).

These fermentation inhibitors usually reduces the ethanol production efficiency significantly when, for example, a weak acid such as acetic acid is present in an amount of about 0.02 mol/L in a fermentation broth, but when the method for producing ethanol of the present invention is used, the ethanol production efficiency is not substantially affected even when a weak acid as a fermentation inhibitor is present in an amount of 0.02 mol/L or more and further efficient ethanol production can be carried out with no problem in the presence of 0.04 mol/L or more of the weak acid. On the other hand, an amount exceeding 0.20 mol/L is likely to affect the ethanol production efficiency, and hence the content is preferably 0.02 mol/L or less, more preferably 0.15 mol/L or less, further preferably 0.12 mol/L or less and particularly preferably 0.08 mol/L or less. Note that the above content is the total content of all weak acids.

Also, furan compounds such as furfural and HMF usually reduce the ethanol production efficiency significantly when present in an amount of about 0.01 mol/L in a fermentation broth, but, when the method for producing ethanol of the present invention is used, the ethanol production efficiency is not substantially affected even when a furan compound as a fermentation inhibitor is present in an amount of 0.01 mol/L or more, and further efficient ethanol production can be carried out with no problem in the presence of 0.02 mol/L or more of the furan compound. On the other hand, an amount exceeding 0.10 mol/L is likely to affect the ethanol production efficiency, and hence the content is preferably 0.10 mol/L or less, more preferably 0.070 mol/L or less, particularly preferably 0.040 mol/L or less. Note that the above content is the total content of all furan compounds.

Accordingly, the method for producing ethanol of the present invention, needless to say, can produce ethanol well under conditions free of weak acids and furan compounds; however, in view of being likely to benefit from an effect of producing ethanol well from a cellulosic biomass hydrolysate even when a fermentation inhibitor is present, it is preferable that a fermentation broth contain from 0.02 mol/L to 0.15 mol/L of weak acids and/or from 0.01 mol/L to 0.10 mol/L of furan compounds. Further, it is more preferable that a fermentation broth contain from 0.04 mol/L to 0.12 mol/L of weak acids and/or from 0.02 mol/L to 0.07 mol/L of furan compounds.

According to the method for producing ethanol of the present invention, the fermentative production of ethanol is carried out using a fermentation broth comprising such a cellulosic biomass hydrolysate and a content of the cellulosic biomass hydrolysate in the fermentation broth can be suitably determined but is more preferably from 0.1 to 20% by mass, further preferably from 0.5 to 15% by mass and particularly preferably from 1 to 10% by mass, in terms of the all monosaccharides based on the total amount in the fermentation broth before supplied into a fermenter. Preferable concentration of each saccharide ranges from 0.1 to 10% by mass, preferably from 0.5 to 5% by mass for xylose, and from 0.0 to 15% by mass, preferably from 0.5 to 5% by mass in total for glucose and other hexoses.

The fermentation broth used for the ethanol production of the present invention may suitably contain other necessary components in addition to a cellulosic biomass hydrolysate. For example, saccharides such as glucose, mannose, galactose and xylose may be contained as a carbon source other than a cellulosic biomass hydrolysate. When these saccharides are additionally contained, a concentration of the monosaccharide is preferably from 0.1 to 10% by mass, more preferably from 1 to 5% by mass, in total with the saccharides derived from a cellulosic biomass hydrolysate. Also, a nitrogen source such as amino acids, urea, polypeptone or nitrogen base without amino acids or yeast extract may be added. Note that, as described later, when a continuous fermentation method is used as the fermentation system, an yeast is also removed when a fermented liquor containing ethanol in a chemostat is removed and consequently the yeast often needs to be grown in a fermenter. For this reason, when the continuous fermentation is carried out over an extended period of time, such a component is preferably contained as necessary so as to be adequate for the yeast growth.

### (3) Fermentation Condition

According to the method for producing ethanol of the present invention, an air supply rate needs to be from 0.0001 to 100 L/hour/g dry cell weight. When a rate is outside this range, the ethanol production efficiency is reduced. The air supply rate is preferably from 0.005 to 100 L/hour/g dry cell weight, more preferably from 0.005 to 10 L/hour/g dry cell weight, preferably from 0.005 to 1.0 L/hour/g dry cell weight. Also, an air supply rate for a batch fermentation method is preferably from 0.005 to 1.0 L/hour/g dry cell weight, more preferably from 0.005 to 0.5 L/hour/g dry cell weight, further preferably from 0.005 to 0.10 L/hour/g dry cell weight, particularly preferably from 0.005 to 0.05 L/hour/g dry cell weight. An air supply rate for a continuous fermentation method is preferably from 0.05 to 100 L/hour/g dry cell weight, more preferably from 0.05 to 10 L/hour/g dry cell weight, further preferably from 0.10 to 1.0 L/hour/g dry cell weight, particularly preferably from 0.10 to 0.5 L/hour/g dry cell weight. The air as used herein means the atmosphere and the amount thereof in terms of the oxygen supply amount is one fifth (⅕) of the amount of the air.

The method for producing ethanol of the present invention may be carried out by the batch fermentation method or the continuous fermentation method but, according to the present invention, it is preferable to carry out the continuous fermentation method because the problems posed in the ethanol production by the continuous fermentation method can be improved. When the present invention is carried out by the continuous fermentation method, it is preferable that a fermentation broth comprising a cellulosic biomass hydrolysate be supplied into a fermenter at a supply rate of from 0.0002 to 2 L/hour/g dry cell weight. The supply rate is preferably from 0.005 to 0.5 L/hour/g dry cell weight, more preferably from 0.01 to 0.05 L/hour/g dry cell weight. Note that in the continuous fermentation method a fermented liquor is removed at the same rate as the supply of a fermentation broth.

In the ethanol fermentation by yeast using a cellulosic biomass hydrolysate, the yeast growth under an aerobic condition in the presence of a cellulosic biomass hydrolysate and the ethanol fermentation by the grown yeast under an anaerobic condition in the presence of a cellulosic biomass hydrolysate are carried out. Thus, in the ethanol production by the batch fermentation method, the step of such an yeast growth and the step of ethanol fermentation by the grown yeast are alternately carried out to employ the condition suitable for each step. However, there is a problem that the ethanol production is discontinued every time the steps are switched.

Whereas, the ethanol production by the continuous fermentation method is advantageous in that it does not involve such a switching step, hence; however, the conditions suitable for the ethanol fermentation are often employed than the conditions suitable for the growth. As a result, the growth of an yeast is suppressed and fails to compensate in a fermenter for the yeast continuously removed as the fermented liquor, thus reducing an yeast concentration, whereby the ethanol production efficiency is also reduced. For this reason, the yeast often needs to be replenished to maintain high ethanol production efficiency when the common continuous fermentation method is employed.

However, according to the method for producing ethanol of the present invention, the growth of an yeast and the ethanol fermentation by the grown yeast are well balanced and thus an efficient ethanol production can be maintained without additional yeast supply during the continuous fermentation.

Further, based on the reason described below, the continuous fermentation is more suitable for the ethanol fermentation by an yeast using a cellulosic biomass hydrolysate and accordingly the method for producing ethanol of the present invention, which enables an efficient ethanol continuous production, is of great significance.

That is, during the growth and fermentation steps, the yeast incorporates fermentation inhibitors such as furfural and HMF in addition to the saccharides present in a cellulosic biomass hydrolysate. These incorporated substances are oxidized and/or reduced and detoxified by enzymes in the yeast cells during the growth or fermentation process. At this process, the enzyme requires a coenzyme (NADH or NADPH), which is produced during the growth or fermentation process (Non Patent Literature 1). For this reason, the batch fermentation in which the growth and ethanol production concentrations fluctuate is not always suitable for the ethanol fermentation by an yeast using a cellulosic biomass hydrolysate. By contrast, in the continuous fermentation, the growth and ethanol production concentrations are maintained substantially in a constant level, which accordingly stabilizes the production concentration of a coenzyme. Therefore, the coenzyme is not excessively or insufficiently supplied and thus the enzyme is insusceptible to fermentation inhibitors, and as a result it is possible to produce ethanol efficiently.

From such a viewpoint, the continuous fermentation method is more preferable for the ethanol production method of the present invention when performed under the conditions of an air supply rate of preferably from 0.05 to 100 L/hour/g dry cell weight, further preferably from 0.05 to 10 L/hour/g dry cell weight, further preferably from 0.10 to 1.0 L/hour/g dry cell weight and supplying the fermentation broth comprising a cellulosic biomass hydrolysate into a fermenter at a supply rate of from 0.005 to 0.5 L/hour/g dry cell weight, further preferably from 0.010 to 0.05 L/hour/g dry cell weight.

The fermentation conditions other than above may be suitably determined but preferable examples of the conditions may be as follows.

The yeast concentration during the ethanol production is preferably adjusted to from 0.5 to 5% by mass on a dry cell weight basis. In the batch fermentation method, the yeast concentration may be adjusted to the above concentration at the point of growth step before the ethanol fermentation step. In the continuous fermentation method, a pre-cultured yeast before initiation of the culture is inoculated to the concentration within the above range or the yeast may be grown about twice the concentration after the inoculation, and the yeast concentration during the ethanol production may be adjusted to within the above range by adjusting the supply rate of the fermentation broth comprising a cellulosic biomass hydrolysate (i.e., the removal rate of the fermented liquor) and culture conditions such as an oxygen concentration.

The temperature during the ethanol production is preferably from 20 to 35° C.

The following conditions are employed to carry out the pre-culture performed before the growth step in the batch fermentation method and the growth step in the continuous fermentation method and the batch fermentation method, and the culture performed to adjust a cell volume to a preferable concentration before the ethanol production in the continuous fermentation method. Usable medium contains a cellulosic biomass hydrolysate as a carbon source, glucose and at least one saccharide selected from the group consisting of mannose, galactose and xylose and further, as necessary, a nitrogen source suitable for the viability such as amino acids, urea, polypeptone or nitrogen base without amino acids and an yeast extract. A concentration of monosaccharide is preferably from 0.1 to 10% by mass, more preferably from 1 to 5% by mass, in total, but in the case where a cellulosic biomass hydrolysate is used as a carbon source, the hydrolysate is used in a volume of preferably 20% by volume or less, more preferably from 10% by volume or less, of the medium volume. The temperature is preferably from 10° C. to 37° C., more preferably from 25° C. to 30° C. The pH is preferably from 4 to 7, more preferably from 4.5 to 6.5. Further, when the pre-culture is carried out under an aerobic condition, a pH is more preferably from 5 to 6.

The efficient ethanol production according to the present invention means that a fermentation yield of 70% by mass or more is achieved within 24 hours after the initiation of fermentation for the batch fermentation method, and that a fermentation yield of 70% by mass or more is maintained even after 24 hours from the initiation of fermentation for the continuous fermentation method.

### Example 1

Hereinafter, the present invention is further described in details with reference to Examples but is not limited thereto.

### Reference Example 1

Using, as a parental strain, yeast Candida intermedia “NBRC10601” deposited in National Institute of Technology and Evaluation, International Patent Organisms Depositary (NITE), 4-6-4T2 strain was obtained by acclimation and natural mutation in accordance with the following procedure.

First, the pH of an aqueous acetic acid solution containing 1% by mass of glucose and xylose respectively was adjusted to 5.0 by magnesium hydroxide, and 20% of the obtained solution and 80% of liquid medium (yeast extract: 1%, yeast nitrogen base without amino acids: 2%) were mixed. 1% of xylose was added to 10 mL of the mixed solution, a platinum loop of yeast Candida intermedia “NBRC10601” was inoculated and cultured at 30° C. for 3 days to obtain a culture broth.

Subsequently, 50% each of an aqueous acetic acid solution containing 1% by mass each of glucose and xylose and adjusted to pH 5.0 in the same manner as above and the liquid medium were mixed, 100 μL of the above culture broth cultured for 3 days was added to 10 mL of this mixed solution and further cultured for 7 more days. Further, 80% of an aqueous acetic acid solution containing 1% by mass each of glucose and xylose and adjusted to pH 5.0 in the same manner as above and 20% of medium were mixed, and 100 μL of the above culture broth cultured for 7 days was added to 10 mL of the mixed solution to further culture 30 more days and obtained an acclimated strain solution.

The above acclimated strain solution was diluted 1000 times and applied to YNB agar medium (glucose: 5%, yeast extract: 1%, yeast nitrogen base without amino acids: 2%, agar: 2%) and cultured at 25° C. for 4 days to subsequently obtain the colonized strain.

The above obtained strain was applied to YNB agar medium (trehalose: 2%, yeast extract: 1%, yeast nitrogen base without amino acids: 2%, agar: 2%), cultured at 25° C. for 3 days, and subsequently the colony formations were confirmed whereby the culture was stored at 4° C. The colonies grown at 4° C. were selected and subjected to an ethanol production test using a phosphate buffer (xylose: 2.5%, KH₂PO₄: 0.1M, pH=5.0, MgSO₄.7H₂O: 0.006M) to select strains having a higher ethanol production ability than that of the parental strain.

The intended yeast was selected in this manner and designated as Candida intermedia 4-6-4T2 strain. The strain was deposited in National Institute of Technology and Evaluation, International Patent Organism Depositary (NITE) under the registration number of FERM BP-11509.

### Example 1

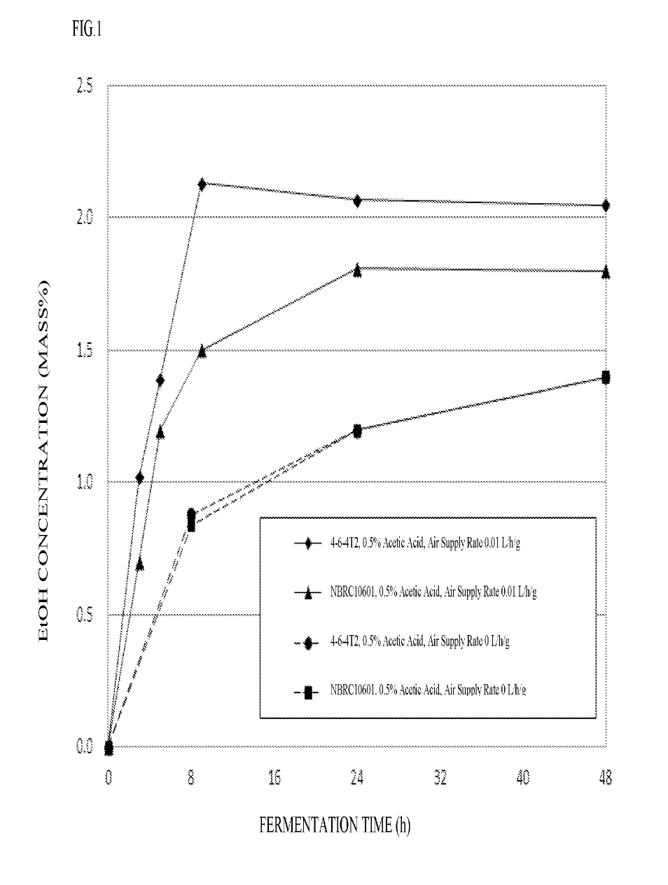

Candida intermedia NBRC10601 or Candida intermedia 4-6-4T2 (FERM BP-11509) was added to YNB medium (glucose 2% by mass and xylose 1% by mass, 2% yeast nitrogen base (free of amino acids) and 1% yeast extract) and pre-cultured at 30° C. for 48 hours at pH 5.5 to 6 (not adjusted). Subsequently, the cell was added to an imitated fermentation broth comprising a cellulosic biomass hydrolysate (glucose 3% by mass, xylose 2% by mass, acetic acid 0.5% by mass (0.08 mol/L), a 0.05 M phosphate buffer, pH 5.5) so as to be 2% by mass (equivalent to dry cell weight). Using this, the ethanol production was carried out at an air supply volume of 0.01 L/h/g dry cell weight and measured time dependent changes in the ethanol concentrations. The results are shown in FIG. 1.

Note that the above imitated fermentation broth comprising a cellulosic biomass hydrolysate of Example 1 contains glucose and xylose, which are representative saccharides in the cellulosic biomass hydrolysate and acetic acid as a fermentation inhibitor in the cellulosic biomass hydrolysate to imitate the fermentation broth comprising a cellulosic biomass hydrolysate.

Further, this ethanol production was carried out without supplying the fermentation broth or removing the fermented liquor, more specifically the ethanol production was carried out by the batch fermentation method, not by the continuous fermentation method. Furthermore, a comparative ethanol production was carried out at an air supply of 0 L/hour/g dry cell weight.

### Example 2

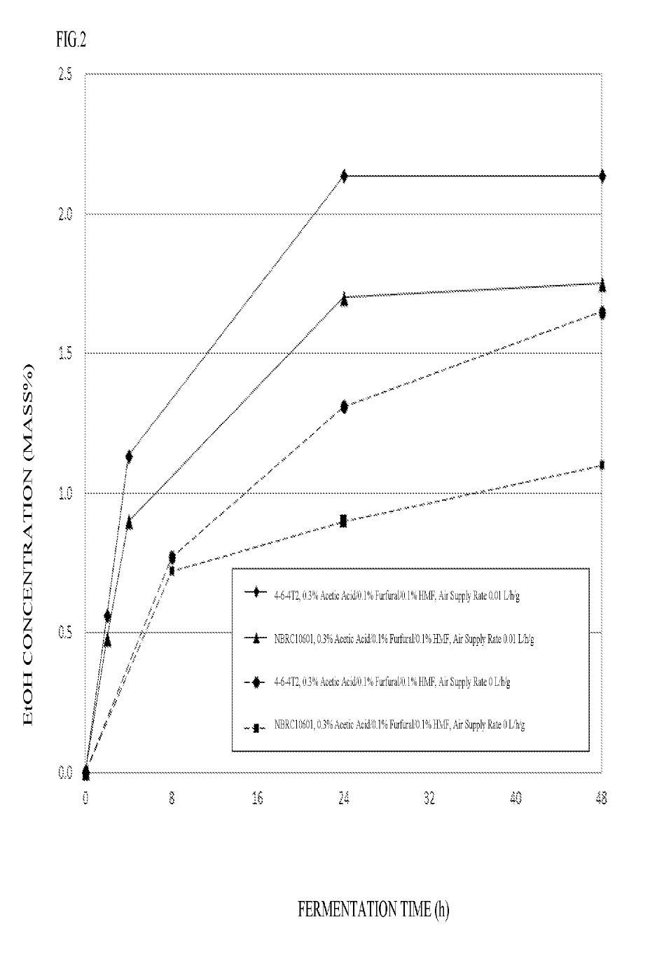

Candida intermedia NBRC10601 or Candida intermedia 4-6-4T2 (FERM BP-11509) was pre-cultured under the same conditions as Example 1 and subsequently added to an imitated fermentation broth comprising a cellulosic biomass hydrolysate (glucose 3%, xylose 2%, acetic acid 0.3% by mass (0.05 mol/L), furfural 0.1% by mass (0.010 mol/L), 5-hydroxymethylfurfural (HMF) 0.1% by mass (0.008 mol/L), a 0.05 M phosphate buffer, pH 5.5), so as to be 2% by mass (equivalent to dry cell weight). Using this, the ethanol production was carried out at an air supply volume of 0.01 L/h/g dry cell weight and measured time dependent changes in the ethanol concentrations. The results are shown in FIG. 2.

Note that the above imitated fermentation broth comprising a cellulosic biomass hydrolysate of Example 2 contains glucose and xylose, which are representative saccharides in the cellulosic biomass hydrolysates and acetic acid, furfural and HMF as fermentation inhibitors in the cellulosic biomass hydrolysate to imitate the fermentation broth comprising a cellulosic biomass hydrolysate.

Further, this ethanol production was carried out without supplying the fermentation broth or removing the fermented liquor, more specifically the ethanol production was carried out by the batch fermentation method, not by the continuous fermentation method. Furthermore, a comparative ethanol production was carried out at an air supply of 0 L/hour/g dry cell weight.

### Example 3

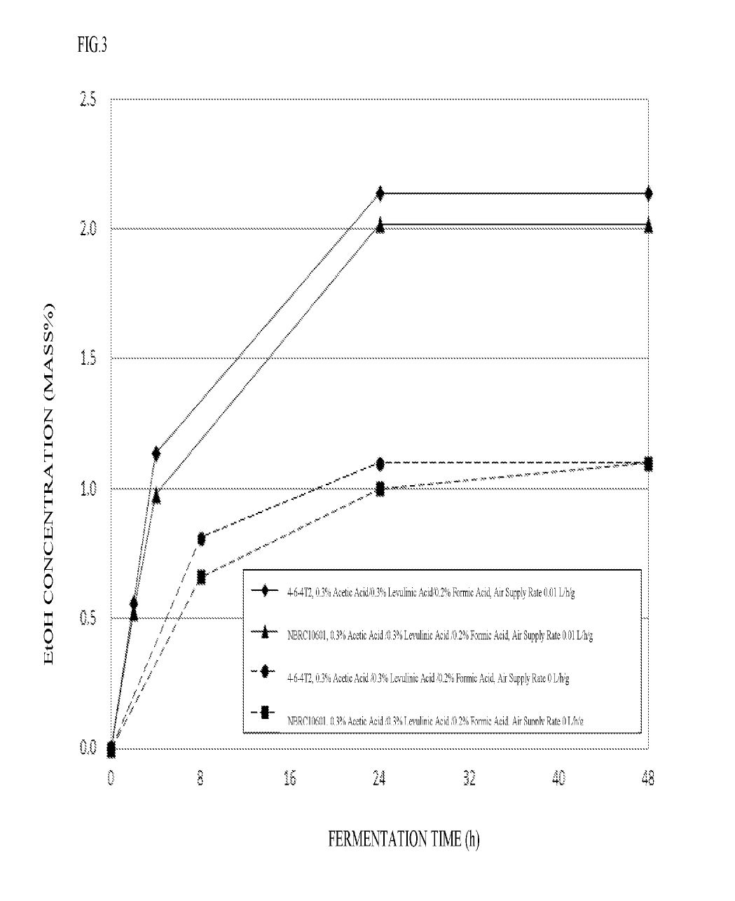

Candida intermedia NBRC10601 or Candida intermedia 4-6-4T2 (FERM BP-11509) was pre-cultured under the same conditions as Example 1 and subsequently added to an imitated fermentation broth comprising a cellulosic biomass hydrolysate (glucose 3% by mass, xylose 2% by mass, acetic acid 0.3% by mass (0.050 mol/L), levulinic acid 0.3% by mass (0.026 mol/L), formic acid 0.2% by mass (0.043 mol/L), a 0.05 M phosphate buffer, pH 5.5) so as to be 2% by mass (equivalent to dry cell weight). Using this, the ethanol production was carried out at an air supply volume of 0.01 L/h/g dry cell weight and measured time dependent changes in the ethanol concentrations. The results are shown in FIG. 3.

Note that the above imitated fermentation broth comprising a cellulosic biomass hydrolysate of Example 3 contains glucose and xylose, which are representative saccharides in the cellulosic biomass hydrolysate and acetic acid, levulinic acid and formic acid as fermentation inhibitors in the cellulosic biomass hydrolysate to imitate the fermentation broth comprising a cellulosic biomass hydrolysate.

Further, this ethanol production was carried out without supplying the fermentation broth or removing the fermented liquor, more specifically the ethanol production was carried out by the batch fermentation method, not by the continuous fermentation method. Furthermore, a comparative ethanol production was carried out at an air supply of 0 L/hour/g dry cell weight.

### Example 4

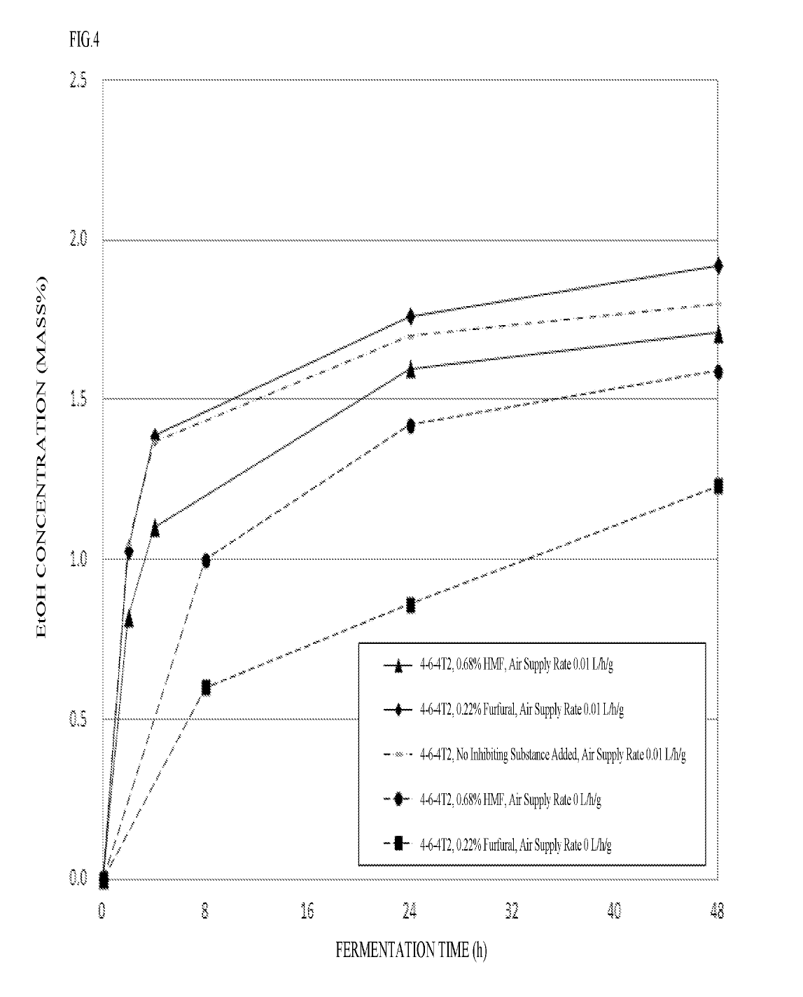

Candida intermedia 4-6-4T2 (FERM BP-11509) was pre-cultured under the same conditions as Example 1 and subsequently added to an imitated fermentation broth comprising a cellulosic biomass hydrolysate (glucose 3% by mass, xylose 2% by mass, furfural 0.22% by mass (0.023 mol/L), a 0.05 M phosphate buffer, pH 5.5) so as to be 2% by mass (equivalent to dry cell weight). Using this, the ethanol production was carried out at an air supply volume of 0.01 L/h/g dry cell weight and measured time dependent changes in the ethanol concentrations. Further, the ethanol productions were carried out respectively under the same conditions except using an imitated fermentation broth comprising a cellulosic biomass hydrolysate in which 0.68% by mass (0.054 mol/L) of 5-hydroxymethylfurfural (HMF) is contained in place of 0.22% by mass of furfural and a fermentation broth containing no fermentation inhibitor as a comparison, and time dependent changes in the ethanol concentrations were measured. The results are shown in FIG. 4.

Note that the above imitated fermentation broth comprising a cellulosic biomass hydrolysate of Example 4 contains glucose and xylose, which are representative saccharides in the cellulosic biomass hydrolysate and furfural or HMF as a fermentation inhibitor in the cellulosic biomass hydrolysate to imitate the fermentation broth comprising a cellulosic biomass hydrolysate.

Further, this ethanol production was carried out without supplying the fermentation broth or removing the fermented liquor, more specifically the ethanol production was carried out by the batch fermentation method, not by the continuous fermentation method. Furthermore, a comparative ethanol production was carried out at an air supply of 0 L/hour/g dry cell weight.

### Example 5

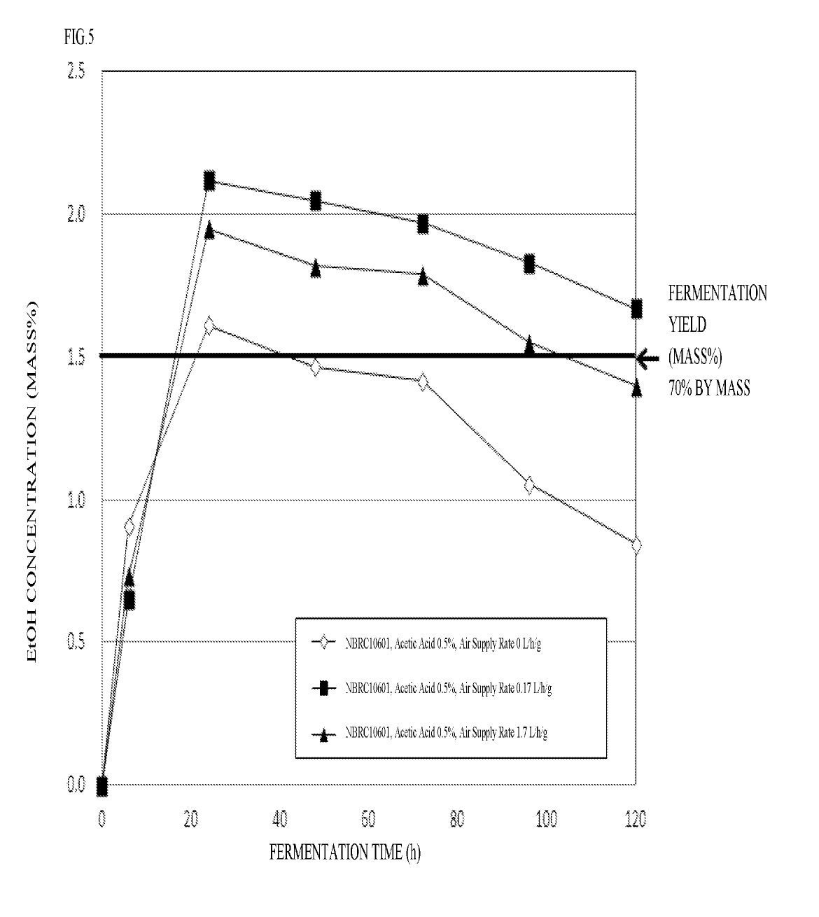

Candida intermedia NBRC10601 was pre-cultured under the same conditions as Example 1 and subsequently added to 0.36 L of an imitated fermentation broth comprising a cellulosic biomass hydrolysate (glucose 3% by mass, xylose 2% by mass, acetic acid 0.5% by mass (0.08 mol/L), a 0.05 M phosphate buffer, pH 5.5) so as to be 2% by mass (7.2 g (dry cell weight)). Using this, the ethanol productions were carried out by the continuous fermentation method at a supply rate of the fermentation broth of 0.015 L/hour and a removal rate of the fermentation broth of 0.015 L/hour and an air supply volume of either 0 L/hour/g dry cell weight, 0.17 L/hour/g dry cell weight or 1.7 L/hour/g dry cell weight, and time dependent changes in the ethanol concentrations were measured. The results are shown in FIG. 5.

Note that the above imitated fermentation broth comprising a cellulosic biomass hydrolysate of Example 5 contains glucose and xylose, which are representative saccharides in the cellulosic biomass hydrolysates and acetic acid as a fermentation inhibitor in the cellulosic biomass hydrolysate to imitate the fermentation broth comprising a cellulosic biomass hydrolysate.

### Example 6

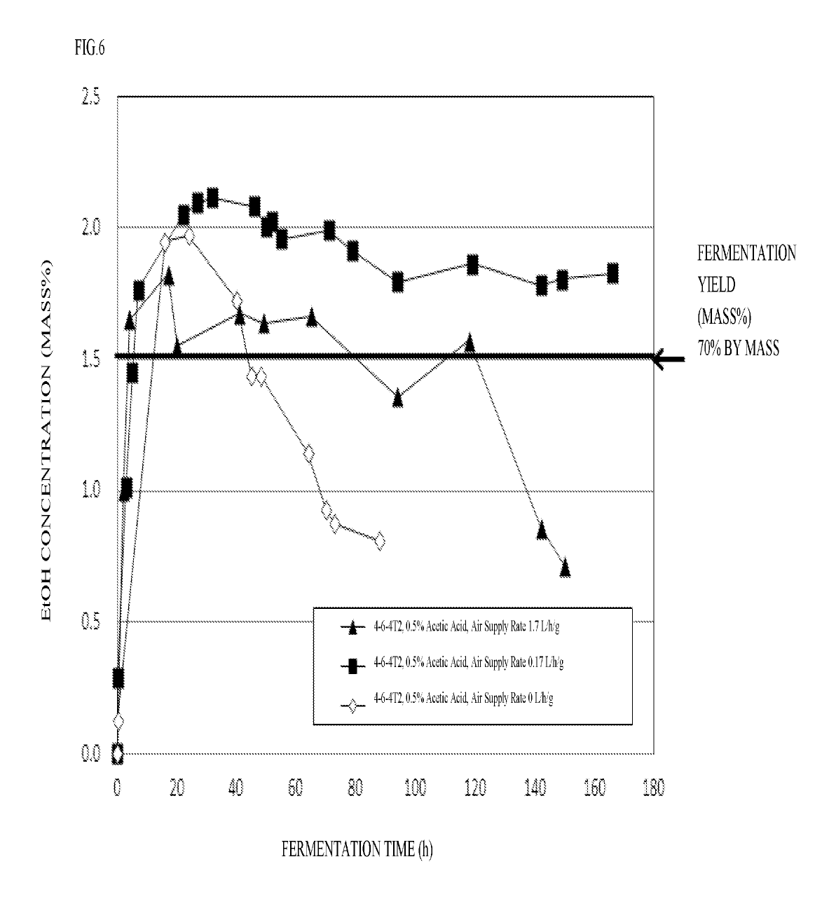

Candida intermedia 4-6-4T2 (FERM BP-11509) was pre-cultured under the same conditions as Example 1 and subsequently added to 0.36 L of an imitated fermentation broth comprising a cellulosic biomass hydrolysate (glucose 3% by mass, xylose 2% by mass, acetic acid 0.5% by mass (0.08 mol/L), a 0.05 M phosphate buffer, pH 5.5) so as to be 2% by mass (equivalent to 7.2 g of dry cell weight). Using this, the ethanol productions were carried out by the continuous fermentation method at a supply rate of the fermentation broth of 0.015 L/hour and a removal rate of the fermentation broth of 0.015 L/hour and an air supply volume of either 0 L/hour/g dry cell weight, 0.17 L/hour/g dry cell weight or 1.7 L/hour/g dry cell weight, and time dependent changes in the ethanol concentrations were measured. The results are shown in FIG. 6.

Note that the above imitated fermentation broth comprising a cellulosic biomass hydrolysate of Example 6 contains glucose and xylose, which are representative saccharides in the cellulosic biomass hydrolysates and acetic acid as a fermentation inhibitor in the cellulosic biomass hydrolysate to imitate the fermentation broth comprising a cellulosic biomass hydrolysate.

The results of FIG. 1 to FIG. 6 reveal that, even when a cellulosic biomass hydrolysate containing a fermentation inhibitor is used as a raw material, an ethanol production efficiency is enhanced by the fermentation performed using an yeast belonging to Candida intermedia under a condition of an air supply rate into a fermenter of from 0.0001 to 100 L/hg/g dry cell weight. Further, the continuous fermentation method is particularly useful.

1. A method for producing ethanol comprising fermenting a fermentation broth comprising a cellulosic biomass hydrolysate using an yeast belonging to Candida intermedia under a condition such that an air supply rate into a fermenter is from 0.0001 to 100 L/hour/g dry cell weight.

2. The method for producing ethanol according to claim 1, wherein the fermentation is continuous fermentation in which the fermentation broth comprising a cellulosic biomass hydrolysate is supplied into the fermenter at a supply rate of from 0.0002 to 2 L/hour/g dry cell weight.

3. The method for producing ethanol according to claim 1, wherein the yeast belonging to Candida intermedia is an yeast designated as 4-6-4T2 and deposited under FERM BP-11509.
